(19) United States
(12) Patent Application Publication
(10) Pub. No.: US 2020/0000946 A1
(43) Pub. Date: Jan. 2, 2020

(54) METHOD AND APPARATUS FOR PREPARING A RADIOLABELED PHARMACEUTICAL

(71) Applicant: The General Hospital Corporation, Boston, MA (US)

(72) Inventors: Daniel L. Yokell, Winthrop, MA (US); Peter A. Rice, North Attleboro, MA (US)

(21) Appl. No.: 16/489,976

(22) PCT Filed: Mar. 2, 2018
(86) PCT No.: PCT/US18/20571

§ 371 Date: Aug. 29, 2019

Related U.S. Application Data
(60) Provisional application No. 62/466,472, filed on Mar. 3, 2017.

(51) Int. Cl. A61K 51/12; A61K 47/02; A61K 51/02; B01J 47/02; G21G 1/10; G21H 5/02

(52) U.S. Cl. CPC A61K 51/121; A61K 47/02; G21H 5/02; B01J 47/02; G21G 1/10; A61K 51/02

(57) ABSTRACT

A method for preparing a radiolabeled pharmaceutical. The method comprises passing a mixture that includes a radiolabeled compound through a column that contains an ion exchange resin to retain the radiolabeled compound on the ion exchange resin. At least a portion of the mixture passes through the column without being retained on the ion exchange resin. The method further comprises eluting the radiolabeled compound off the ion exchange resin using an eluting solution (e.g., a sodium chloride solution) to form a radiolabeled pharmaceutical.

### CROSS-REFERENCES TO RELATED APPLICATIONS

This application claims priority to U.S. patent application Ser. No. 62/466,472 filed Mar. 3, 2017.

### STATEMENT REGARDING FEDERALLY SPONSORED RESEARCH

Not Applicable.

### BACKGROUND OF THE INVENTION

### 1. Field of the Invention

This invention relates to the preparation of a radiolabeled pharmaceutical, specifically an improved method and an apparatus for radiolabeled pharmaceutical preparation.

### 2. Description of the Related Art

Current methods and apparatuses used for the preparation of radiolabeled pharmaceuticals involve manual interventions and manipulations at various instances throughout the preparation process. Current methods and apparatuses may be configured to perform a single production run.

Manual interventions and manipulations increase the risk for operator error during the preparation process. Manual interventions may also increase the time required to complete the process thereby making commercialization more difficult and less efficient. Single production runs produce a small amount of radiolabeled pharmaceutical which may not be commercially applicable.

Therefore, what is needed is an improved method and apparatus for preparing a radiolabeled pharmaceutical, such as ammonia N 13 or sodium fluoride F18.

### SUMMARY OF THE INVENTION

According to one embodiment, the present invention provides a method for preparing a radiolabeled pharmaceutical. The method may comprise (a) passing a mixture including a radiolabeled compound through a column containing an ion exchange resin. The ion exchange resin may retain the radiolabeled compound on the ion exchange resin, and at least a portion of the mixture may pass through the column without being retained on the ion exchange resin. The method may further comprise (b) eluting the radiolabeled compound off the ion exchange resin using an eluting solution to form a radiolabeled pharmaceutical, wherein the eluting solution comprises ions suitable for intravenous infusion into a subject. A “subject” is a mammal, preferably a human.

In some embodiments, the radiolabeled compound may be ammonia N13. The ion exchange resin may be cationic. In other embodiments, the ion exchange resin may be anionic. Step (b) of the method may further comprise using an inert gas under positive pressure to transfer the radiolabeled pharmaceutical through a sterilizing filter.

In some embodiments, before step (a) of the method, the mixture may be passed through an additional column that may contain a second ion exchange resin to remove impurities from the mixture. The second ion exchange resin may be cationic in some embodiments and anionic in other embodiments. In some embodiments, steps (a) and (b) can be repeated. Step (b) may further comprise purifying the radiolabeled pharmaceutical with sterile water. In some embodiments, the solution for injection may be isotonic and the radiolabeled compound may be ammonia N13.

According to another embodiment, the present invention provides an apparatus for preparing a radiolabeled pharmaceutical on a radiolabeled product synthesizer. The radiolabeled product synthesizer may have an outlet, and an inlet for receiving a mixture including a radiolabeled compound. The apparatus may comprise a support and a column attached to the support. The column may contain an ion exchange resin for retaining the radiolabeled compound on the ion exchange resin. The apparatus may further comprise a vessel attached to the support, a conduit in fluid communication with the column and vessel, and an outlet for removal of a radiolabeled pharmaceutical. The vessel attached to the support may contain a an eluting solution comprising ions suitable for intravenous infusion into a subject.

In some embodiments, the apparatus may comprise an additional column containing a second ion exchange resin to remove impurities from the mixture. The additional column may be in fluid communication with the conduit via a second conduit. A valve may be in fluid communication with the conduit and second conduit. The valve may have a first position in which the mixture flows from the additional column to the column, and the valve may have a second position in which the eluting solution (e.g., a sodium chloride solution) flows from the vessel through the conduit to the column. A sterilizing filter may be in fluid communication with the column and the outlet, and may be configured to purify the radiolabeled pharmaceutical. In some embodiments, the radiolabeled compound may be ammonia N13. The ion exchange resin may be cationic in some embodiments and anionic in other embodiments.

The apparatus and method may fully automate the purification and formulation of the radiolabeled pharmaceutical for injection. The radiolabeled pharmaceutical for injection may be ammonia N13, sodium fluoride F18, or any anionic or cationic PET radiopharmaceutical.

The automation allows the elimination of manual interventions in the purification of ammonia N13 injection. The production of ammonia N13 occurs in FDA regulated drug manufacturing environment according to prescribed processes in NDA, ANDA or IND regulatory filings. Elimination of manual manipulations increases cGMP compliances and greatly reduces the potential for operator errors in the purification and formulation of ammonia N13 injection and sodium fluoride F18 injection.

It is therefore an advantage of the invention to provide an improved method and apparatus for preparing a radiolabeled pharmaceutical, such as ammonia N13 and sodium fluoride F18.

These and other features, aspects, and advantages of the present invention will become better understood upon consideration of the following detailed description, drawing and appended claims.

### BRIEF DESCRIPTION OF THE DRAWINGS

FIG. 1 is a schematic diagram of an apparatus for practicing the present invention.

FIG. 2 shows a schematic prototype apparatus that was prepared for preparing a radiolabeled pharmaceutical.

FIG. 3 shows a top view of a schematic prototype of a microfluidic chip for preparing a radiolabeled pharmaceutical.

### DETAILED DESCRIPTION OF THE INVENTION

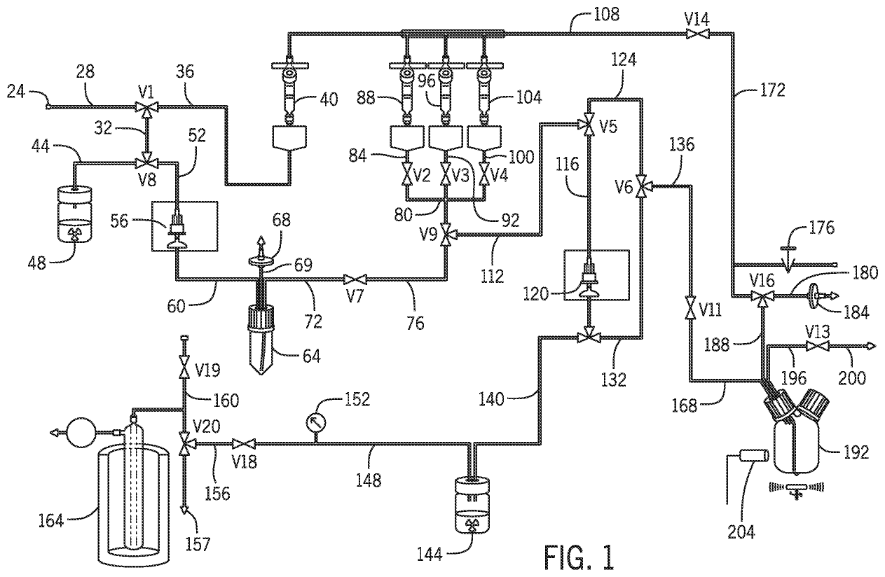

Referring to FIG. 1, an apparatus 10 for preparing a radiolabeled pharmaceutical, such as ammonia N13, sodium fluoride F18, or any anionic or cationic PET radiopharmaceutical is shown. The apparatus 10 may have an inlet 24. The inlet 24 may be configured to supply a mixture including a radiolabeled compound to the apparatus 10. The mixture may be synthesized using known techniques such as those described in U.S. Pat. No. 5,345,477, which is incorporated herein by reference.

The inlet 24 may be connected via a conduit 28 to a valve V1. Conduit 28 may provide fluid communication between the inlet 24 and valve V1. Valve V1 may be connected to a vessel 40 via a conduit 36. Conduit 36 may provide fluid communication between vessel 40 and valve V1. Valve V1 may be connected to a valve V8 via a conduit 32. Valve V1 may be a three way valve or a mixing valve, thereby selectively providing fluid communication between the inlet 24, valve V8, and vessel 40 via conduits 28, 32, and 36 or any combination thereof.

Valve V8 may be connected to a waste vessel 48 via a conduit 44 which provides fluid communication between valve V8 and waste vessel 48. The waste vessel 48 may be configured to receive impurities in the mixture. Valve V8 may be connected to a column 56 via a conduit 52. The column 56 may contain an ion exchange resin. In some embodiments, the ion exchange resin of the column 56 may be anionic. In other embodiments, the ion exchange resin of the column 56 may be anionic. Column 56 may be a solid phase extraction cartridge. A non-limiting example of a cartridge that may be used may be QMA anion solid-phase extraction cartridge produced by Waters. Valve V8 may be a three way valve or a mixing valve, thereby selectively providing fluid communication between valve V1, waste vessel 48, and column 56 via conduits 32, 44, and 52 or any combination thereof.

The column 56 may be connected to a reservoir 64 via a conduit 60, which provides fluid communication between the column 56 and reservoir 64. The reservoir 64 may be configured to receive the mixture and may be vented through an exhaust 68 via a conduit 69. The reservoir 64 may be connected to a valve V7 via a conduit 72. Valve V7 may be connected to a valve V9 via a conduit 76. Valve V7 may be a two way valve, selectively providing fluid communication between reservoir 64 and valve V9 via conduit 72 and conduit 76.

Valve V9 may be a three way valve or a mixing valve that may be connected to a valve V2, a valve V3, and a valve V4 via a conduit 80. Conduit 80 may provide fluid communication between valve V9 and valves V2, V3, and V4. Valve V2 may be a two way valve, and may be connected to a vessel 88 via a conduit 84. Conduit 84 may provide fluid communication between vessel 88 and conduit 94. Vessel 88 may be configured to contain an eluting solution (e.g., a sodium chloride solution). Valve V3 may be a two way valve and may be connected to a vessel 96 via a conduit 92. Conduit 92 may provide fluid communication between valve V3 and vessel 96, which may be configured to contain sterile water. Valve V4 may be a two way valve, and may be connected to a vessel 104 via a conduit 100. Conduit 100 may provide fluid communication between valve V4 and vessel 104, which may be configured to contain sterile water. A conduit 108 connects vessels 88, 96, and 104 to vessel 40 and valve V14. Conduit 108 provides fluid communication between valve V14 and vessels 40, 88, 96,and 104. Valve V9 may be connected to a valve V5 via a conduit 112 and thereby selectively provides fluid communication between valve V7 and valves V2, V3, and V4 via conduits 76 and 80. Valve V9 further provides selective fluid communication between valve V7 and valve V5 via conduits 76 and 112. Valve V9 also provides selective fluid communication between valve V5 and valves V2, V3, and V4.

Valve V5 may be a three way or mixing valve that may be connected to a column 120 via a conduit 116, which provides fluid communication between column 120 and valve V5. The column 120 may contain an ion exchange resin. In some embodiments, the ion exchange resin of the column 120 may be anionic. In other embodiments, the ion exchange resin of the column 120 may be cationic. Column 120 may be a solid phase extraction cartridge. A non-limiting example of a cartridge that may be used may be CM solid-phase cation extraction cartridge produced by Waters that is configured to retain cationic ammonia N13. The column 120 may be connected to a valve V10 via a conduit 128, which provides fluid communication between column 120 and valve V10. Valve V5 may be connected to a valve V6 via a conduit 124, which provides fluid communication between valves V5 and V6. Valve V5 thereby selectively provides fluid communication between valve V9 and valve V6 via conduits 112 and 124. Valve V5 further provides selective fluid communication between valve V9 and column 120 via conduits 112 and 116. Valve V5 selectively provides fluid communication between column 120 and valve V6.

Valve V10 may be a three way valve or a mixing valve that may be connected to a waste vessel 144 via a conduit 140. Conduit 140 provides fluid communication between waste vessel 144 and conduit 140. The waste vessel 144 may be connected to a valve V18 and a pressure indicator 152 via a conduit 148. Conduit 148 provides fluid communication between the waste vessel 144, pressure indicator 152, and a valve V18. Valve V18 may be a two way valve. Valve V10 thereby provides selective fluid communication between column 120 and waste vessel 144 via conduit 140. Valve V10 further provides selective fluid communication between column 120 and valve V6 via conduit 132. Valve V10 also provides selective fluid communication between valve V6 and waste vessel 144 via conduits 132 and 140.

Valve V18 may be connected to a valve V20 via a conduit 156, which provides fluid communication between valves V18 and V20. Valve V20 may be a three way or a mixing valve that may be connected to an exhaust 157, a valve V19 and a vacuum pump 164. A conduit 160 connects valve V20 to valve V19 and the vacuum pump 164, and provides fluid communication between valves V19, V20, and the vacuum pump 164. Valve V20 selectively provides fluid communication between exhaust 157, vacuum pump 164, and valves V18 and V19 via conduits 156 and 160. Valve V19 may be a two way valve.

Valve V6 may be a three-way or a mixing valve that may be connected to valve V10 via conduit 132, which provides fluid communication between valve V6 and valve V10. Valve V6 may be connected to a valve V11 via a conduit 136, which provides fluid communication between valves V6 and V11. Valve V6 may thereby provide selective fluid communication between valves V5, V10, and V11.

Valve V11 may be a two way valve that may be connected to vessel 192 via a conduit 168, which may provide fluid communication between the vessel 192 and valve V11. Vessel 192 may be connected to a valve V13 via a conduit 196, which may provide fluid communication between the vessel 192 and valve V13. Valve V13 may be a two way valve that may be connected to an outlet 200. In some embodiments, the outlet may be an isolator that may be an ISO Class 5 isolator. A vessel indicator 204 may be connected to the vessel 192.

Valve V14 may be connected to a pressure regulator 176 and a valve V16 via a conduit 172, which may provide fluid (e.g., inert gas) communication between valve V14, pressure regulator 176, and valve V16. Valve V14 may be a two way valve that may selectively provide fluid communication between vessels 40, 88, 96,104, valve V16, and pressure regulator 176. Valve V16 may be a three way valve that may be connected to an exhaust 184 via a conduit 180, which may provide fluid communication between exhaust 184 and valve V16. Valve V16 may be connected to vessel 192 via a conduit 188, which provides fluid communication between vessel 192 and valve V16. Valve V16 may be a three-way or a mixing valve that may thereby provide selective fluid communication between valve V14, exhaust 184, and vessel 192.

Now that the components of the apparatus 10 have been described, the operation and functionality of the apparatus 10 may be appreciated. The mixture may be synthesized using known techniques such as those described in U.S. Pat. No. 5,345,477, which is incorporated herein by reference. In some embodiments, the mixture supplied to the inlet 24 may include a radiolabeled compound. A non-limiting example of a radiolabeled compound that may be included in the mixture is ammonia N13, which may be an active pharmaceutical ingredient. As described in U.S. Pat. No. 5,345,477, the cyclotron run may be initiated and 16.5 MeV protons may be used to irradiate the target solution for a period of time using a range of beam currents. In some embodiments, the period of time may be 25 to 50 minutes and the beam current can range from 15-25 μAmp. The mixture produced under the aforementioned conditions is expected to be between 174 mCi and 813 mCi. The pharmaceutical ingredient may be synthesized in a cyclotron target to be transferred to the apparatus 10 as a mixture via inlet 24.

In some embodiments, inlet 24 is configured to receive the mixture from a cyclotron target and introduce the mixture to the apparatus 10. The mixture may be pushed from the inlet 24 through valve V1 via conduit 28. Vessel 40 may be configured to contain a gas, which may be an inert gas. Non-limiting examples of the gas that may be contained in vessel 40 include nitrogen, helium, or argon. Vessel 40 may be provided with overpressure from pressure regulator 176 via valve V14 and conduits 172 and 108. Vessel 40 may be configured to apply a positive gas overpressure through conduit 36 to push the mixture through valves V1 and V8, into column 56 via conduits 32 and 52.

Valve V1 may be configured to be in a first position where the mixture can flow from the inlet 24 through valve V1 via conduit 28 to valve V8 via conduit 32. In the first position of valve V1, pressure applied from vessel 40 may push the mixture through valve V1 to valve V8. Valve V8 may be in a first position in order to allow the mixture to flow through valve V8 into column 56 via conduit 52. The inlet 24 could also be configured to deliver the liquid from a vial as well in situations where the user is unable to connect directly to the cyclotron. The vial would contain the same material that the cyclotron would deliver.

In some embodiments, column 56 may comprise an ion exchange resin. The ion exchange resin in column 56 may be a strong anion exchange resin. The ion exchange resin in column 56 can be configured to remove anionic impurities in the mixture, such impurities can be transferred to waste vessel 48 via conduit 52, valve V8, and conduit 44. A non-limiting example of an impurity to be removed by the column 56 is fluorine 18 aqueous. Fluid communication between column 56 and waste vessel 48 may occur when valve V8 is in a second position. The second position of valve V8 may allow impurities retained on the ion exchange resin to be pushed though valve V8 via conduit 52 into waste vessel 48 via conduit 44.

In other embodiments, the column 56 may comprise a strong cationic exchange resin. Column 56 can be configured to remove cationic impurities present in the mixture via the strong cationic exchange resin. Column 56 may be single use and disposable.

The mixture may be transferred from the column 56 to the reservoir 64, which is configured to receive the mixture. In some embodiments, the reservoir 64 can be vented via conduit 69 and exhaust 68. Vessel 40 may be configured to apply a positive gas overpressure through conduit 36 to push the mixture through valves V1 and V8, though column 56 via conduits 32 and 52 into reservoir 64 via conduit 60. Vessel 40 may also apply a positive gas overpressure through conduit 36 to push waste into waste vessel 48 via valves V1 and V8 and conduits 28, 32, 44, and 52. In other embodiments, waste may be transferred from inlet 24 to waste vessel 48 via valves V1 and V8 and conduits 28, 32, and 44.

Once transfer of the mixture into reservoir 64 is complete, vacuum pump 164 may apply a negative pressure on the apparatus 10 thereby applying a negative pressure on the reservoir 64. Negative pressure draws the mixture out of reservoir 64 and pulls it into column 120 via conduit 116 from reservoir 64 through valve V7 via conduit 72, through valve V9 via conduit 76, and through valve V5 via conduit 112. Pressure indicator 152 may be configured to indicate the pressure applied by the vacuum pump 164 as noted in conduit 148. It is to be appreciated that although pressure indicator 152 is positioned in conduit 148, one or more pressure indicators similar to pressure indicator 152 may be positioned anywhere throughout the apparatus 10.

In some embodiments, transfer of the mixture from reservoir 64 to column 120 may be provided by a first position of valves V9, V5, V10, and V20. The first position of valve V9 provides fluid communication between valve V7 and valve V5 via conduits 76 and 112. The first position of valve V5 may provide fluid communication between valve V9 and column 120 via conduits 112 and 116. The first position of valve V10 may provide fluid communication between column 120 and waste vessel 144 via conduits 128 and 140. The first position of valve V20 provides fluid communication between vacuum pump 164 and waste vessel 144 via conduits 160, 156, 148 and valve V18.

In some embodiments, column 120 may comprise an ion exchange resin. The ion exchange resin in column 120 may be a strong cationic exchange resin. In other embodiments, the ion exchange resin in column 120 may be a strong anionic exchange resin. The ion exchange resin may be configured to selectively retain the radiolabeled compound in column 120 while allowing the remaining mixture to be transferred to waste vessel 144 through valve V10 via conduits 128 and 140. Valve V10 may remain in the first position to provide fluid communication between column 120 and waste vessel 144.

Column 120 with the selectively retained radiolabeled compound may be eluted with an eluting solution that may be configured to remove the radiolabeled compound and formulate the radiolabeled compound into a solution for injection. The eluting solution may be contained in vessel 88 and may be transferred to column 120 via conduit 116 through valve V2 via conduit 82, valve V9 via conduit 80, and valve V5 via conduit 112. The elution of the radiolabeled compound may be provided by an open position of valve V2 and a second position of valve V9 that provides fluid communication between vessel 88 and valve V5 via valve V2 and conduits 84, 80, and 112. Valve V5 may remain in the first position in order to provide the eluting solution (e.g., a sodium chloride solution) to the column 120 via conduit 116. Vessel 88 may be provided with overpressure from pressure regulator 176 via valve V14 and conduits 172 and 108. The eluting solution may be pushed by the overpressure applied to the vessel 88. Valves V3 and V4 may be in a closed position while the elution process occurs such that sterile water may not enter conduit 80.

In other embodiments, the column 120 may comprise a strong anionic exchange resin. Column 120 can be configured to remove anionic impurities present in the mixture via the strong anionic exchange resin. Column 120 may be single use and disposable.

The eluting solution can be a sodium chloride solution. The sodium chloride solution can have a concentration of 0.1 wt. % to 23.5 wt. %. The sodium chloride solution may have a concentration of 0.1 wt. % to 2.0 wt. %, USP, or 0.5 wt. % to 1.5 wt. %, or 0.7 wt. % to 1.1 wt. %, or 0.9 wt. %. The eluting solution may further comprise a salt selected from the group consisting of potassium chloride, calcium chloride, sodium lactate, and mixtures thereof. The eluting solution may further comprise a buffering agent selected from the group consisting of phosphate salts (e.g., sodium phosphate or potassium phosphate), acetate salts (e.g., sodium acetate or potassium acetate), citrate salts (e.g., sodium citrate or potassium citrate), and mixtures thereof. The eluting solution can be isotonic with respect to blood plasma. By isotonic with respect to blood plasma, we mean having an osmolarity of about 270 mOsm/L to about 310 mOsm/L.

In one non-limiting embodiment, the column 120 is eluted with 0.9 wt. % sodium chloride for injection, USP, which removes the ammonia N13 and formulates the ammonia N13 into an isotonic solution for injection.

The solution for injection may be transferred to vessel 192. In some embodiments, the solution may be transferred to vessel 192 during the elution of the radiolabeled compound from the ion exchange resin. The solution can be transferred from the column 120 to vessel 192 via conduit 168 through valve V10 via conduit 128, through valve V6 via conduit 132, and through valve V11 via conduit 136. Valve V10 may be in a second position to provide fluid communication between column 120 and valve V6. Vessel indicator 204 may be configured to indicate the amount of solution in the vessel 192. Valve V6 may be in a first position that may provide fluid communication between valve V10 and vessel 192 via conduits 132, 136, 168 and valve V11.

In some embodiments, the solution for injection may be transferred from vessel 192 through a sterilizing filter. A non-limiting range of sterilizing filters that could be used is 0.10 μ-0.90 μ. Positive gas overpressure may be used to transfer the solution from the vessel 192 through the sterilizing filter. Alternatively, a vacuum could be used to transfer the solution from the vessel 192 through the sterilizing filter. In some embodiments, an inert gas can be utilized in the transfer of the solution, the solution may include 13N-NH₄ or any radiolabeled pharmaceutical. Passing the solution for injection through the sterilizing filer can allow the radiolabeled pharmaceutical to pass through the sterilizing filter. In some embodiments, the radiolabeled pharmaceutical may be a sterile injectable ammonia N13 drug product. The radiolabeled pharmaceutical may be transferred to the outlet 200 through valve V13, which may be placed in an open position, via conduit 196. In some embodiments, the outlet 200 may be connected to an isolator.

Pressure regulator 176 may be configured to regulate the pressure applied throughout the apparatus 10. Pressure regulator 176 may be configured to regulate and adjust the pressure applied to vessels 40, 88, 96, and 104 thereby controlling the speed at which fluids can communicate within the apparatus 10 as well as the force applied through the apparatus 10.

In some embodiments, it may be desirable to perform multiple production runs of the radiolabeled pharmaceutical through the apparatus 10. In some embodiments, the radiolabeled pharmaceutical may be 13N ammonia. In other embodiments, the radiolabeled pharmaceutical may be 18F sodium fluoride. In still other embodiments, the radiolabeled pharmaceutical may be any radiochemical agent that may be desirable for use in in medical studies such as heart studies. This can be achieved with repeating the steps described above after further addition of the mixture from the cyclotron target and introduction of the mixture to the apparatus 10 at inlet 24.

Prior to performing each production run, the apparatus 10 may be flushed with sterile water. Sterile water can be applied to apparatus from vessels 96 and 104 concurrently or independently. Valve V3 may be in an open position in order to provide sterile water from vessel 96 to valve V9 via conduits 92 and 80. Valve V4 may be in an open position in order to provide sterile water from vessel 104 to valve V9 via conduits 100 and 80. Valve V9 may be in a third position to provide fluid communication from valve V2 to valves V5 and V7. Valve V5 may be placed in a second position to provide fluid communication between valve V9, column 120, and valve V6. Valve V10 may be placed in a third position that provides fluid communication between column 120, waste vessel 144 and valve V6. Valve V6 may be placed in a second position that may provide fluid communication between valves V5, V10, and V11. Valve V8 may be placed in a third position, thereby providing fluid communication between valve V7, reservoir 64, column 56, waste vessel 48, and valve V1. Valve V1 may be placed in a second position that provides fluid communication between vessel 40, valve V8, and inlet 24 via conduits 36, 32, and 28.

The completion of a production run may be defined as the completion of sterile filtration where the solution for injection has been passed through the sterilizing filter. Quality and purity testing may be performed on samples of the solution for injection. Quality testing may include thin layer chromatography.

The process described above for the production of ammonia N13 injection may be possible on other automated radiochemistry synthesis units which are cassette based. Non-limiting examples are the GE Medical System FASTIab, GE Medical System FASTIab2, GE Tracerlab MX, Ora Neptis synthesis modules, Siemens Explora one, Scintomics GRP, IBA Synthera, Trasis AllinONE, Eckert and Ziegler Modular Lab Pharmtracer, or other cassette based synthesis units.

The process described above for the production of ammonia N13 injection may be possible on automated radiochemistry synthesis units which are tubing based. Non-limiting examples are the GE Tracerlab FX-FN, GE Tracerlab FX2N, GE Tracerlab FX-M, GE Tracerlab FX2-M, Eckert and Ziegler Modular Lab, Synthera, Ora Seed, Siemens Explora, or other tubing based synthesis units.

For a system to be able to automate the ammonia N13 injection process as described above, the system should have the ability to provide an overpressure of inert gas, such as nitrogen, argon or helium, ability to create vacuum, have holders for two solid phase extraction cartridges and contain vessels for containing the necessary reagents of sterile water for injection and sterile eluting solution (e.g., a sodium chloride solution) for injection.

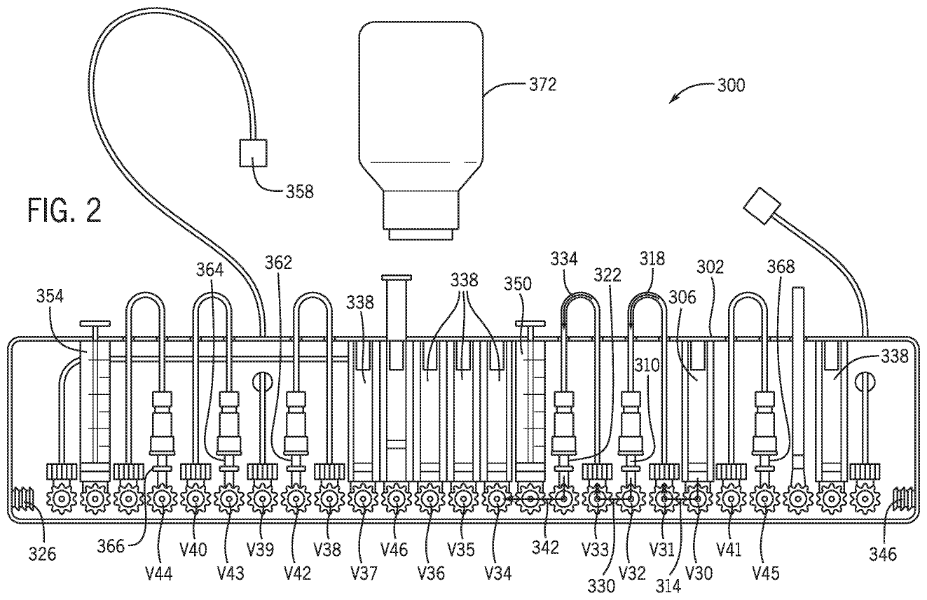

FIG. 2 shows an apparatus 300 prepared as a prototype for preparing a radiolabeled pharmaceutical. The apparatus 300 was configured to complete multiple production runs of a radiolabeled pharmaceutical. In some embodiments, the radiolabeled pharmaceutical may be 13N ammonia. In other embodiments, the radiolabeled pharmaceutical may be 18F sodium fluoride. In still other embodiments, the radiolabeled pharmaceutical may be any radiochemical agent that may be desirable for use in in medical studies such as heart studies. The apparatus 300 had an inlet 302 configured to supply a mixture including a radiolabeled compound to the apparatus 300. The mixture including 13N ammonia was contained in a reservoir 306 and was generated using a cyclotron. The mixture can be transferred from the reservoir 306 to an anion exchange column 310 via valves V30, V31 and conduits 314 and 318. The anion exchange column 310 may be configured to remove anionic impurities in the mixture prior to passing the mixture to a cation exchange column 322, such impurities can be transferred to a waste output 326. The mixture can be passed from the anion exchange column 310 to the cation exchange column 322 via valves V32, V33 and conduits 330 and 334.

The cation exchange column 322 may comprise a cation exchange resin. The ion exchange resin in the cation exchange column 322 may be a strong cationic exchange resin. The ion exchange resin may be configured to selectively retain the radiolabeled compound in the cation exchange column 322 while allowing the remaining mixture to be transferred to the waste output 326.

The cation exchange column 322 with the selectively retained radiolabeled compound was eluted with an eluting solution comprising sodium chloride solution to remove the radiolabeled compound from the cation exchange column 322 and formulate the radiolabeled compound into a solution for injection. The sodium chloride solution may be contained in vessels 338 which may be transferred to the cation exchange column 322 via valves V34, V35, V36, V37, and conduit 342. Vessels 338 were provided with pressure from a gas supply 346, and syringes 350, 354 were available for pressure control. The radiolabeled pharmaceutical was transferred from the cation exchange column 322 to the output 358 of the apparatus 300.

As depicted in FIG. 2, additional cation exchange columns 362, 364, 366, 368 were positioned on the apparatus 300. The additional cation exchange columns 362, 364, 366, 368 were provided to allow for multiple production runs of the radiolabeled pharmaceutical without operator intervention. The additional cation exchange columns feature input valves V38, V39, V40, V41 and output valves V42, V43, V44, V45. The input valve V38, V39, V40, V41 and output valve V42, V43, V44, V45 remain closed while the corresponding additional cation exchange column 362, 364, 366, 368 is not in use on the apparatus 300. When the corresponding additional cation exchange column 362, 364, 366, 368 is in use, the input valve V38, V39, V40, V41 and output valve V42, V43, V44, V45 may be opened such that fluid communication is provided through the additional cation exchange column 362, 364, 366, 368. The input valves V38, V39, V40, V41 and output valves V42, V43, V44, V45 may be operated manually or automatically such that fluid communication is provided to one cation exchange column 362, 364, 366, 368 for each production run, and fluid communication is restricted from the other cation exchange columns.

Prior to performing each production run, the apparatus 300 may be flushed with sterile water that was contained in vessel 372 which was placed in fluid communication with the apparatus 300 when valve V46 was opened following each production run.

In another embodiment, an apparatus for preparing a radiolabeled pharmaceutical can have a surface defining a microfluidic channel having an inlet for receiving a mixture including a radiolabeled compound and having an outlet for removal of a radiolabeled pharmaceutical. The apparatus can have an ion exchange resin positioned in the microfluidic channel, the ion exchange resin retaining the radiolabeled compound on the ion exchange resin. A vessel can be in fluid communication with the microfluidic channel, the vessel containing an eluting solution comprising ions suitable for intravenous infusion into a subject. In some embodiments. a second ion exchange resin to remove impurities from the mixture can be positioned in the microfluidic channel downstream of the ion exchange resin. The radiolabeled compound can be ammonia N13, and the eluting solution can be sodium chloride solution. The sodium chloride solution can have a concentration of 0.1 wt. % to 23.5 wt. % or 0.1 wt. % to 2.0 wt. %, in non-limiting examples. The eluting solution can further comprise a salt that can be selected from the group consisting of potassium chloride, calcium chloride, sodium lactate, and mixtures thereof. In some embodiments, the eluting solution can further comprise a buffering agent. The buffering agent can be selected from the group consisting of phosphate salts, acetate salts, citrate salts, and mixtures thereof. The eluting solution can be isotonic with respect to blood plasma, and the ion exchange resin can be cationic or anionic.

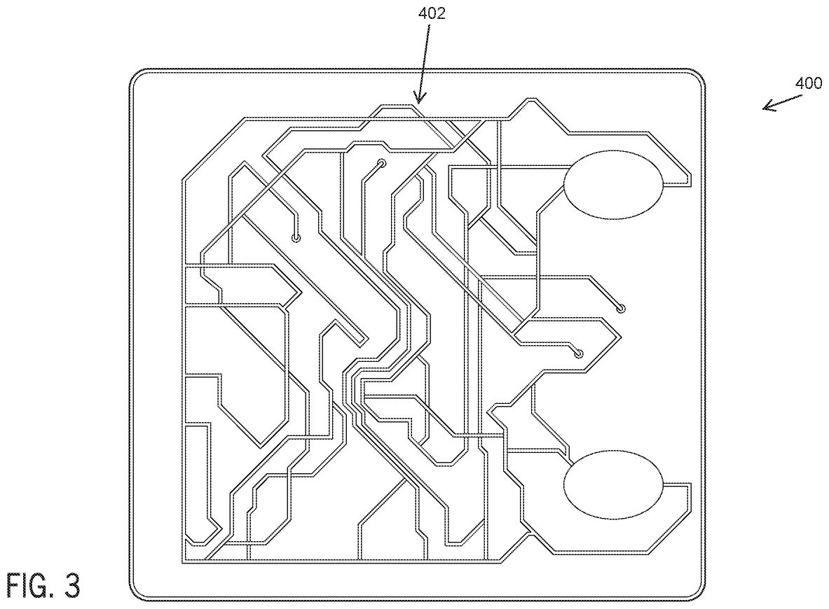

FIG. 3 shows a top view of a schematic prototype of a microfluidic chip 400 for preparing a radiolabeled pharmaceutical, according to an aspect of the disclosure. The process described above for the production of ammonia N13 injection may be possible on automated radiochemistry synthesis units on a microfluidic chip such as the microfluidic chip 400. The microfluidic chip 400 can have a set of micro-channels 402 etched or molded into a material (e.g. glass, silicon or polymer such as PDMS, for polydimethylsiloxane). The micro-channels 402 can form the microfluidic chip 400 and can be connected together in order to achieve the process described above for the production of ammonia N13 injection. The network of micro-channels embedded in the microfluidic chip 400 can have inputs and outputs pierced through the chip that provide liquids (or gases) that can be injected and removed from the microfluidic chip 400 (through tubing, syringe adapters, simple holes in the chip, etc.). A microfluidic channel, as the terms is used herein, typically has a channel width perpendicular to a longitudinal axis of the channel (i.e., a path along which fluid flows during ordinary operation) that is about 1 millimeter or smaller.

Although fluid flow has been described in considerable detail with reference to certain embodiments, it is to be appreciated that established fluid communication between elements may allow fluid flow between those components in some embodiments.

Thus, the invention provides an improved method and apparatus for preparing a radiolabeled pharmaceutical, such as ammonia N13.

Although the invention has been described in considerable detail with reference to certain embodiments, one skilled in the art will appreciate that the present invention may be practiced by other than the described embodiments, which have been presented for purposes of illustration and not of limitation. Therefore, the scope of the appended claims should not be limited to the description of the embodiments contained herein.

What is claimed is:

1. A method for preparing a radiolabeled pharmaceutical, the method comprising:
(a) passing a mixture including a radiolabeled compound through a column containing an ion exchange resin to retain the radiolabeled compound on the ion exchange resin, where at least a portion of the mixture passes through the column without being retained on the ion exchange resin; and
(b) eluting the radiolabeled compound off the ion exchange resin using an eluting solution to form a radiolabeled pharmaceutical, the eluting solution comprising ions suitable for intravenous infusion into a subject.

2. The method of claim 1 wherein:
the radiolabeled compound is ammonia N13.

3. The method of claim 1 wherein:
the radiolabeled compound is sodium fluoride F18.

4. The method of claim 1 wherein:
the eluting solution is a sodium chloride solution.

5. The method of claim 4 wherein:
the sodium chloride solution has concentration of 0.1 wt. % to 23.5 wt. %.

6. The method of claim 4 wherein:
the sodium chloride solution has concentration of 0.1 wt. % to 2.0 wt. %.

7. The method of claim 4 wherein:
the eluting solution further comprises a salt selected from the group consisting of potassium chloride, calcium chloride, sodium lactate, and mixtures thereof.

8. The method of claim 1 wherein:
the eluting solution further comprises a buffering agent.

9. The method of claim 8 wherein:
the buffering agent is selected from the group consisting of phosphate salts, acetate salts, citrate salts, and mixtures thereof.

10. The method of claim 1 wherein:
the eluting solution is isotonic with respect to blood plasma.

11. The method of claim 1 wherein:
the ion exchange resin is cationic.

12. The method of claim 1 wherein:
the ion exchange resin is anionic.

13. The method of claim 1 wherein:
step (b) further comprises using a positive inert gas pressure to transfer the radiolabeled pharmaceutical through a sterilizing filter.

14. The method of claim 1 further comprising:
before step (a), passing the mixture through an additional column containing a second ion exchange resin to remove impurities from the mixture.

15. The method of claim 14 wherein:
the second ion exchange resin is cationic.

16. The method of claim 14 wherein:
the second ion exchange resin is anionic.

17. The method of claim 14 further comprising:
repeating steps (a) and (b).

18. The method of claim 1 wherein:
step (b) further comprises purifying the radiolabeled pharmaceutical with sterile water.

19. An apparatus for preparing a radiolabeled pharmaceutical on a radiolabeled product synthesizer having an outlet and an inlet for receiving a mixture including a radiolabeled compound, the apparatus comprising:
a support;
a column attached to the support, the column containing an ion exchange resin for retaining the radiolabeled compound on the ion exchange resin;
a vessel attached to the support, the vessel containing an eluting solution comprising ions suitable for intravenous infusion into a subject;
a conduit in fluid communication with the column and vessel; and
an outlet for removal of a radiolabeled pharmaceutical.

20. The apparatus of claim 19 further comprising:
an additional column containing a second ion exchange resin to remove impurities from the mixture,
wherein the additional column is in fluid communication with the conduit via a second conduit.

21. The apparatus of claim 19 further comprising:
a valve in fluid communication with the conduit and second conduit,
wherein the valve has a first position in which the mixture flows from the additional column to the column, and the valve has a second position in which the sodium chloride solution flows from the vessel through the conduit to the column.

22. The apparatus of claim 19 further comprising:
a sterilizing filter in fluid communication with the column and the outlet,
wherein the sterilizing filter is configured to purify the radiolabeled pharmaceutical.

23. The apparatus of claim 19 wherein:
the radiolabeled compound is ammonia N13.

24. The apparatus of claim 19 wherein:
the eluting solution is a sodium chloride solution.

25. The apparatus of claim 24 wherein:
the sodium chloride solution has concentration of 0.1 wt. % to 23.5 wt. %.

26. The apparatus of claim 24 wherein:
the sodium chloride solution has concentration of 0.1 wt. % to 2.0 wt. %.

27. The apparatus of claim 24 wherein:
the eluting solution further comprises a salt selected from the group consisting of potassium chloride, calcium chloride, sodium lactate, and mixtures thereof.

28. The apparatus of claim 19 wherein:
the eluting solution further comprises a buffering agent.

29. The apparatus of claim 28 wherein:
the buffering agent is selected from the group consisting of phosphate salts, acetate salts, citrate salts, and mixtures thereof.

30. The apparatus of claim 19 wherein:
the eluting solution is isotonic with respect to blood plasma.

31. The apparatus of claim 19 wherein:
the ion exchange resin is cationic.

32. The apparatus of claim 19 wherein:
the ion exchange resin is anionic.

33. An apparatus for preparing a radiolabeled pharmaceutical, the apparatus comprising:
a surface defining a microfluidic channel having an inlet for receiving a mixture including a radiolabeled compound and having an outlet for removal of a radiolabeled pharmaceutical;
an ion exchange resin positioned in the microfluidic channel, the ion exchange resin retaining the radiolabeled compound on the ion exchange resin; and
a vessel in fluid communication with the microfluidic channel, the vessel containing an eluting solution comprising ions suitable for intravenous infusion into a subject.

34. The apparatus of claim 33 further comprising:
a second ion exchange resin to remove impurities from the mixture,
wherein the second ion exchange resin is positioned in the microfluidic channel downstream of the ion exchange resin.

35. The apparatus of claim 33 wherein:
the radiolabeled compound is ammonia N13.

36. The apparatus of claim 33 wherein:
the eluting solution is a sodium chloride solution.

37. The apparatus of claim 36 wherein:
the sodium chloride solution has concentration of 0.1 wt. % to 23.5 wt. %.

38. The apparatus of claim 36 wherein:
the sodium chloride solution has concentration of 0.1 wt. % to 2.0 wt. %.

39. The apparatus of claim 33 wherein:
the eluting solution further comprises a salt selected from the group consisting of potassium chloride, calcium chloride, sodium lactate, and mixtures thereof.

40. The apparatus of claim 39 wherein:
the eluting solution further comprises a buffering agent.

41. The apparatus of claim 40 wherein:
the buffering agent is selected from the group consisting of phosphate salts, acetate salts, citrate salts, and mixtures thereof.

42. The apparatus of claim 33 wherein:
the eluting solution is isotonic with respect to blood plasma.

43. The apparatus of claim 33 wherein:
the ion exchange resin is cationic.

44. The apparatus of claim 33 wherein:
the ion exchange resin is anionic.
